(19) United States
(12) Patent Application Publication
(10) Pub. No.: US 2020/0196905 A1
(43) Pub. Date: Jun. 25, 2020

(54) SYSTEM AND METHOD FOR REAL-TIME TRACKING OF A PROBE DURING A PROCEDURE

(71) Applicants: Guanbo CHEN, Coralville, IA (US); Mahta MOGHADDAM, Los Angeles, CA (US); Pratik SHAH, Vista, CA (US); John STANG, Los Angeles, CA (US); University of Southern California, Los Angeles, CA (US)

(72) Inventors: Guanbo Chen, Los Angeles, CA (US); Mahta Moghaddam, Los Angeles, CA (US); Pratik Shah, Los Angeles, CA (US); John Stang, Los Angeles, CA (US)

(21) Appl. No.: 16/641,115

(22) PCT Filed: Aug. 21, 2018
(86) PCT No.: PCT/US18/47229

§ 371 Date: Feb. 21, 2020

Related U.S. Application Data
(60) Provisional application No. 62/548,680, filed on Aug. 22, 2017.

(51) Int. Cl. A61B 5/06; A61B 5/05; A61B 5/00; A61B 34/10; A61B 34/20

(52) U.S. Cl. CPC A61B 5/061; A61B 5/0507; A61B 5/743; A61B 2034/2046; A61B 34/20; A61B 2034/107; A61B 2034/2074; A61B 34/10

(57) ABSTRACT

Systems and methods for tracking a probe during a procedure. In one embodiment, the method includes monitoring a position of the probe in an imaging region during the procedure using microwave inverse scattering and contrast source inversion. The method also includes solving for a contrast source in the imaging region using compressive sensing and group sparsity. The contrast source exists at a surface of the probe or within the probe. The method further includes imaging the contrast source and the probe by solving a linear inverse scattering problem with a group sparsity constraint. The method also includes determining a location of the probe in the imaging region during the procedure based on the imaging of the contrast source and the probe. The method further includes displaying an image of the location of the probe relative to an anatomy feature in the imaging region during the procedure.

### CROSS-REFERENCE TO RELATED APPLICATIONS

This application is a non-provisional of and claims benefit of U.S. Provisional Application No. 62/548,680, filed on Aug. 22, 2017, the entire contents of which are incorporated herein by reference.

### TECHNICAL FIELD

This disclosure relates to a probe. This disclosure also relates to a procedure (for example, a medical procedure). This disclosure further relates to a method for tracking a probe during a procedure. This disclosure also relates to methods for using microwave imaging to locate and monitor the position of a probe during a medical treatment, diagnosis, and/or biopsy.

### BACKGROUND OF THE INVENTION

In recent years, thermal ablation has seen increased use in the treatment of various diseases. In the case of thermal ablation, magnetic resonance (MR), X-ray computed tomography (CT) or ultrasound techniques have been used to guide an ablation probe to the treatment region and monitor the probe's movement. However, the high expenses and complexity of magnetic resonance and the harmful ionizing nature of X-ray computed tomography have limited their clinical usage. The more widely used ultrasound-guided system has a relatively lower cost. However, the ultrasonic-guided system is a hand-held device and can only generate a two-dimensional (2D) monitoring image which provides limited information.

### SUMMARY OF THE INVENTION

Accordingly, embodiments described herein relate to real-time tracking of a probe during a procedure using inverse scattering. For example, an inverse scattering method with compressive sensing may be used to image and track an ablation probe during an interstitial medical procedure. A contrast source inversion (CSI) method may be used to solve the inverse scattering problem, which determines the location of the probe by utilizing the scattered field data produced by the contrast source current at the surface of the probe. Though the contrast sources at the surface of the probe may be different for different transmitters, they may all sparse signals within an imaging region and have the same shape and structure. Thus, a fast spectral gradient projection method and a separable approximation method may be used to solve the linear inverse problem with the group sparsity constraints and reconstruct the surface profile of the probe. A vector network analyzer-based inverse scattering measurement system may be used to acquire scattered data from the probe. A graphics processing unit (GPU) may be used to accelerate the inversion process and achieve real-time monitoring.

The disclosure provides a method for tracking a probe during a procedure. In one embodiment, the method includes monitoring a position of the probe in an imaging region during the procedure using microwave inverse scattering and contrast source inversion. The method also includes solving for a contrast source in the imaging region using compressive sensing and group sparsity. The contrast source exists at a surface of the probe or within the probe. The method further includes imaging the contrast source and the probe by solving a linear inverse scattering problem with a group sparsity constraint. The method also includes determining a location of the probe in the imaging region during the procedure based on the imaging of the contrast source and the probe. The method further includes displaying an image of the location of the probe relative to an anatomy feature in the imaging region during the procedure.

The disclosure also provides a system for tracking a probe during a procedure. In one embodiment, the system includes a plurality of antennas and a computer. The plurality of antennas are arranged in a three-dimensional array in an imaging region. The plurality of antennas are configured to generate a contrast source by exciting a medium of the probe during the procedure. The computer is operatively connected to the plurality of antennas. The computer includes an electronic processor and a display screen. The electronic processor is configured to determine scattering data generated by the contrast source based on a plurality of measurements from the plurality of antennas. The electronic processor is also configured to determine a linear inverse scattering solution including a group sparsity constraint based on the scattering data. The electronic processor is further configured to image the contrast source and the probe based on the linear inverse scattering solution. The electronic processor is also configured to determine a location of the probe in the imaging region during the procedure based on the imaging of the contrast source and the probe. The display screen is configured to display the location of the probe relative to an anatomy feature in the imaging region during the procedure.

Some features of some embodiments of the disclosure include: 1) using microwave inverse scattering and contrast source inversion to monitor the probe during a treatment; 2) using compressive sensing and group sparsity to solve for the contrast source at the surface of the probe; and 3) using a spectral gradient-projection method and a separable approximation method to solve the linear inverse scattering problem with group sparsity constraint to image the contrast source and the probe.

Other aspects of various embodiments will become apparent by consideration of the detailed description and accompanying drawings.

### BRIEF DESCRIPTION OF THE DRAWINGS

The drawings are of illustrative embodiments. They do not illustrate all embodiments. Other embodiments may be used in addition or instead. Details that may be apparent or unnecessary may be omitted to save space or for more effective illustration. Some embodiments may be practiced with additional components or steps and/or without all of the components or steps that are illustrated. When the same numeral appears in different drawings, it refers to the same or like components or steps.

FIG. 1 is a diagram of a tracking system for tracking a probe during a procedure, in accordance with some embodiments.

FIG. 2 is an image of an ablation probe, in accordance with some embodiments.

FIG. 3 is a diagram of an exemplary probe inserted into a brain lesion.

FIG. 4 is a flowchart of a method for tracking a probe during a procedure, in accordance with some embodiments.

FIG. 5 is a diagram of an imaging region, in accordance with some embodiments.

FIG. 6 is an exemplary imaging result of a probe inserted into human head model.

FIG. 7 is an exemplary imaging result of a metallic probe.

FIG. 8 is an exemplary imaging result of a dielectric (i.e., non-metallic) probe.

FIG. 9 is an image of a vector network analyzer-based inverse scattering measurement system, in accordance with some embodiments.

FIG. 10 is an exemplary comparison of multi-parameter images reconstructed with l_{1,2} group sparsity regularization (2nd column), l₁ sparsity regularization (3rd column) and l₂ Tikhonov regularization (4th column), in accordance with some embodiments. The parameter images of Δε_{∞}, Δε_{δp}, and of Δε_{σ}, are shown in the 1st, 2nd and 3rd rows respectively.

### DETAILED DESCRIPTION

One or more embodiments are described and illustrated in the following description and accompanying drawings. These embodiments are not limited to the specific details provided herein and may be modified in various ways. Furthermore, other embodiments may exist that are not described herein. Also, the functionality described herein as being performed by one component may be performed by multiple components in a distributed manner. Likewise, functionality performed by multiple components may be consolidated and performed by a single component. Similarly, a component described as performing particular functionality may also perform additional functionality not described herein. For example, a device or structure that is “configured” in a certain way is configured in at least that way, but may also be configured in ways that are not listed. Furthermore, some embodiments described herein may include one or more electronic processors configured to perform the described functionality by executing instructions stored in non-transitory, computer-readable medium. Similarly, embodiments described herein may be implemented as non-transitory, computer-readable medium storing instructions executable by one or more electronic processors to perform the described functionality.

In addition, the phraseology and terminology used herein is for the purpose of description and should not be regarded as limiting. For example, the use of “including,” “containing,” “comprising,” “having,” and variations thereof herein is meant to encompass the items listed thereafter and equivalents thereof as well as additional items. The terms “connected” and “coupled” are used broadly and encompass both direct and indirect connecting and coupling. Further, “connected” and “coupled” are not restricted to physical or mechanical connections or couplings and can include electrical connections or couplings, whether direct or indirect. In addition, electronic communications and notifications may be performed using wired connections, wireless connections, or a combination thereof and may be transmitted directly or through one or more intermediary devices over various types of networks, communication channels, and connections. Moreover, relational terms such as first and second, top and bottom, and the like may be used herein solely to distinguish one entity or action from another entity or action without necessarily requiring or implying any actual such relationship or order between such entities or actions.

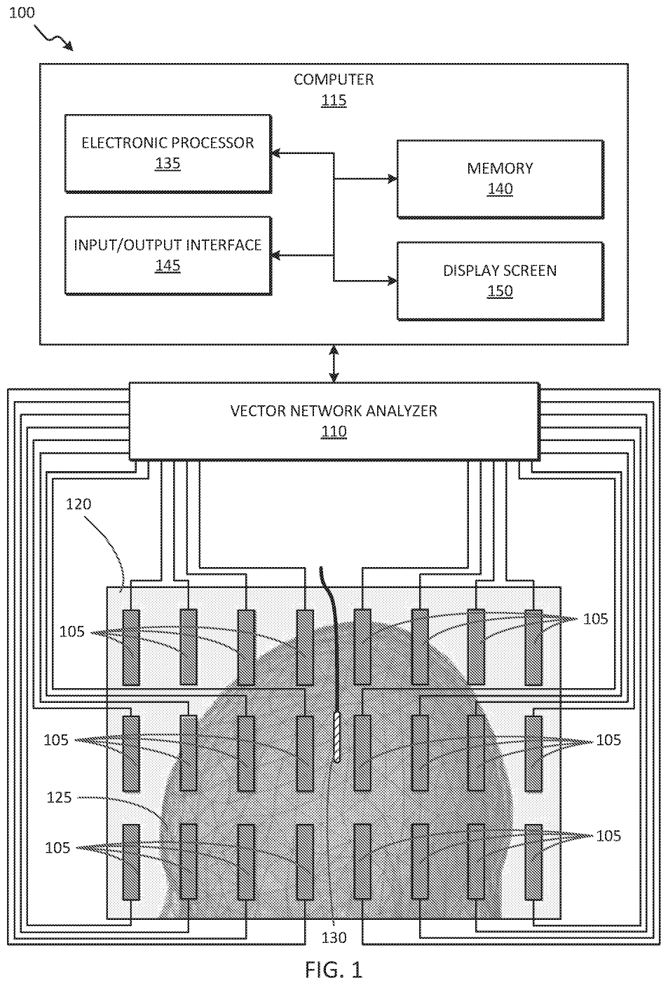

FIG. 1 is a diagram of one example embodiment of a tracking system 100. In the embodiment illustrated in FIG. 1, the tracking system 100 includes antennas 105, a vector network analyzer 110, and a computer 115. In alternate embodiments, the tracking system 100 may include fewer or additional components in configurations different from the configuration illustrated in FIG. 1. The plurality of antennas 105 are arranged in a three-dimensional array in an imaging region 120. In the embodiment illustrated, a head 125 of a subject and a probe 130 are positioned in the imaging region 120. The probe 130 is positioned within the head 125. For example, the subject may be undergoing a medical procedure in which the probe 130 is positioned with the head 125 of the subject for thermal ablation. As will be described in more detail below, the tracking system 100 locates and monitors the position of the probe 130 during a procedure (for example, a medical treatment, a diagnosis, or a biopsy).

In some embodiments, the probe 130 is a medical procedure probe. For example, the probe 130 may be a medical treatment (therapy) probe, a biopsy probe, a diagnostic probe, or a combination thereof. In some embodiments, the probe 130 is a thermal treatment (ablation) probe. In some embodiments, the probe 130 includes a metallic material. For example, the probe 130 may include steel, aluminum, copper, or a combination thereof. Alternatively or in addition, the probe 130 includes a non-metallic material. For example, the probe 130 may include a dielectric material such as Teflon. Alternatively or in addition, the probe 130 includes a biocompatible material. For example, the probe 130 may include a ceramic, a metal, a polymer, or a combination thereof.

The antennas 105 are configured to generate a contrast source by exciting a medium (for example, a metal or dielectric) of the probe 130 during the procedure. The antennas 105 include one or more transmitting antennas that are configured to transmit an excitation signal into the imaging region 120 to produce an electric field in the imaging region 120. In some embodiments, the contrast source is defined as a product of a material contrast and a total electric field in the imaging region 120. The antennas 105 also include one or more receiving antennas that are configured to measure scattering parameters in the imaging region 120. In the embodiment illustrated in FIG. 1, the antennas 105 are operatively connected directly to the vector network analyzer 110. Alternatively or in addition, the antennas 105 are operatively connected to the vector network analyzer 110 via one or more switching matrices (not shown). Alternatively or in addition, the antennas 105 are operatively connected to the computer 115 (either directly or via one or more switching matrices).

In some embodiments, the vector network analyzer 110 includes components or combinations of different components, including all or some of the various components described below with respect to the computer 115.

In the embodiment illustrated in FIG. 1, the computer 115 includes an electronic processor 135 (for example, a microprocessor, or other electronic controller), memory 140, an input/output interface 145, a display screen 150, and a bus. In alternate embodiments, the computer 115 may include fewer or additional components in configurations different from the configuration illustrated in FIG. 1. In some embodiments, the electronic processor 135 includes one or more graphics processing units (GPUs). The bus connects various components of the computer 115 including the memory 140 to the electronic processor 135. The memory 140 includes read only memory (ROM), random access memory (RAM), an electrically erasable programmable read-only memory (EEPROM), other non-transitory computer-readable media, or a combination thereof. The electronic processor 135 is configured to retrieve program instructions and data from the memory 140 and execute, among other things, instructions to perform the methods described herein. Alternatively, or in addition to, the memory 140 is included in the electronic processor 135.

The input/output interface 145 includes routines for transferring information between components within the computer 115 and other components of the tracking system 100, as well as components external to the tracking system 100. The input/output interface 145 is configured to transmit and receive signals via wires, fiber, wirelessly, or a combination thereof. Signals may include, for example, information, data, serial data, data packets, analog signals, or a combination thereof.

The display screen 150 displays various information of the tracking system 100. For example, the display screen 150 may display an image of the location of the probe 130. The display screen 150 is a suitable display, for example, a liquid crystal display (“LCD”) screen, a lighting-emitting diode (“LED”) screen, or an organic LED (“OLED”) screen. In some embodiments, the display screen 150 includes a touch screen display. In some embodiments, the display screen 150 is separated from the computer 115.

In some embodiments, the electronic processor 135 determines scattering data generated by the contrast source based on a plurality of measurements from the antennas 105. For example, in some embodiments, the vector network analyzer 110 determines a plurality of scattering parameters (indicating a scattered electric field measured in the imaging region 120) based on the plurality of measurements from the antennas 105. The vector network analyzer 110 sends the determined plurality of scattering parameters to the electronic processor 135 as scattering data (for example, via the input/output interface 145). In some embodiments, the vector network analyzer 110 links a plurality of total electric fields in the imaging region 120 generated by one or more transmitting antennas to the plurality of scattering parameters measured at one or more receiving antennas with an arbitrary inhomogeneous imaging background.

In some embodiments, the electronic processor 135 determines a linear inverse scattering solution including a group sparsity constraint based on the scattering data. In some embodiments, the electronic processor 135 images the contrast source and the probe 130 based on the determined linear inverse scattering solution. In some embodiments, the electronic processor 135 determines the linear inverse scattering solution using a spectral gradient projection and separable approximation. In some embodiments, the electronic processor 135 determines a location of the probe 130 in the imaging region 120 during the procedure based at least in part on the imaging of the contrast source and the probe 130. In some embodiments, the electronic processor 135 also determines a three-dimensional (3D) shape of the probe 130 during the procedure based at least in part on the imaging of the contrast source and the probe 130. In some embodiments, the determined location of the probe 130 relative to an anatomy feature in the imaging region 120 is displayed on the display screen 150 during the procedure. For example, the determined location of the probe 130 relative to a brain tumor may be displayed on the display screen 150.

In some embodiments, the tracking system 100 implements a microwave-based probe tracking method, which uses the scattered field generated by the contrast source of the probe 130 to reconstruct the 3D shape and location of the probe 130 during a procedure. In some embodiments, the procedure is a medical procedure. For example, the procedure is a medical treatment (for example, a thermal therapy), a diagnosis, or a biopsy.

The tracking system 100 implements the contrast source inversion (CSI) method [1] to solve the linear inverse scattering problem. The tracking system 100 implements a fast spectral gradient projection method to solve the local optimization problem with sparsity constraint. The microwave probe tracking method implemented by the tracking system 100 can be validated by imaging a pulsed eddy current (PEC) probe in a realistic interstitial medical procedure numerical model.

Method

In some embodiments, in solving the inverse scattering problem for tracking the probe 130, the scattered electric field is measured in the form of scattering parameters (S-parameters) by the vector network analyzer 110. The volume integral equation (VIE) [2] is used to link the contrast source of the probe to the measured scattered S-parameters, which can be written as:

S_{m,n}^{scat}(ω,t)=k_{b}²∫∫∫G_{m,n}(r,r′)Eₙ(r′)·O(ω,r′)dv′ (1)

where r′ is the position vector in the imaging region, Eₙ is the total field in the imaging region 120 due to transmitter n, k_{b} is the lossless background wavenumber, and O is the dielectric contrast function. The dielectric contrast function, O, can be written as:

$$O ( ω , {r}^{′} ) = \frac{1}{{ϵ}_{b}} [ {Δϵ}_{inf} ( {r}^{′} ) + \frac{{Δϵ}_{δ p} ( {r}^{′} )}{1 + j ω r} + \frac{Δ σ ( {r}^{′} )}{j {ωϵ}_{0}} ] \qquad ( 2 )$$

where Δ∈_{inf} is the contrast permittivity at infinite frequency, Δ∈_{δp} is the contrast differential permittivity at zero frequency and infinite frequency, and Δσ(r) is contrast conductivity with regard to the imaging background. The vector G̅_{m,n}, is the waveport numerical vector Green's function [2] which links the total electric fields in the imaging region 120 excited by the transmitting antenna n to the S-parameters measured at the antenna m with an arbitrary inhomogeneous imaging background.

In solving this inverse scattering problem, the contrast source inversion method can be used to solve for the contrast source. In some embodiments, the contrast source is defined as the product of the material contrast and total electric field in the imaging region 120. The contrast source in the imaging region 120 generated by exciting transmitting antenna n can be written as,

xₙ(r′)=O(r′)·Eₙ(r′) (3)

where r′ is the position vector of the imaging region 120 and xₙ(r′) is one row of the matrix X̅(r′, n). The matrix X̅(r′, n) can be expressed as,

\hat{X}(r′,n)=[\hat{X}_{∈inf}(r′,n)\hat{X}_{∈δp}(r′,n)\hat{X}_{σ}(r′,n)] (4)

By only solving for the contrast source, the non-linear inverse scattering problem is reduced to a linear problem, which can be written as,

Ŷ=Ā\hat{X}_{∈inf}+Ā\hat{X}_{∈δp}+Ā\hat{X}_{σ} (5)

where matrix X̅ is the scattered S-parameters, and matrix Ā contains the waveport numerical Green's function and the background wavenumber. X̅_{εinf}, X̅_{εδp}, and X̅_{σ} are the contrast source matrices with material parameter ε_{inf}, ε_{δp}, and σ, respectively.

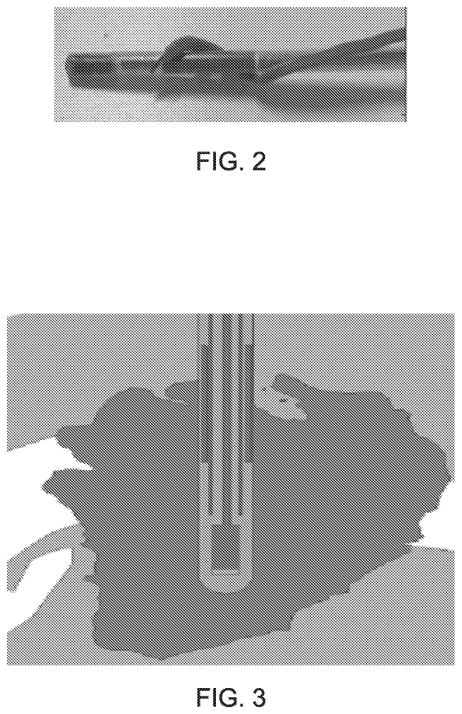

In some embodiments, a 1.9 GHz ablation or guidance probe, as shown in FIG. 2, is used to perform the medical procedure. For example, FIG. 3 illustrates an exemplary ablation probe inserted into a brain lesion. Within the imaging region 120, if the probe is metallic, the contrast source is only present at the surface of the probe 130. If the probe 130 is not metallic, the contrast source is only present at the location of the probe 130 within the imaging region 120. For example, the contrast source may only be present within the probe 130. Both scenarios yield a group sparse solution of the contrast source within the imaging region 120. It means the zeros elements of each column of matrix X̅ should typically be at the same location or with the same index. The linear contrast source inversion can be formulated as a lasso problem [4] with a least squares error function plus a mixed (1, 2) norm sparsity constraint, which can be written as,

J(X̅)=∥Y̅−ĀX̅∥₂²+λ∥X̅∥_{1,2} (6)

where J(x) is the cost function, and is a regularization parameter. In some embodiments, the tracking system 100 implements a fast spectral projected gradient method [5] and a separate approximation for sparse reconstruction method [6] to solve for this lasso problem with group sparsity constraints and to generate sparse solutions for the contrast source at the surface of the probe 130. Then, the 3D profile and the location of the probe 130 can be reconstructed. In some embodiment, during the reconstruction process, GPU parallel computing is utilized to accelerate the solving of the linear inverse problem. GPU parallel computing can provide a refresh rate of approximately one frame per second. For example, in some embodiments, the computer 115 uses one or more graphics processing units (GPUs) (included in some implementations of the electronic processor 135) to solve the linear inverse problem.

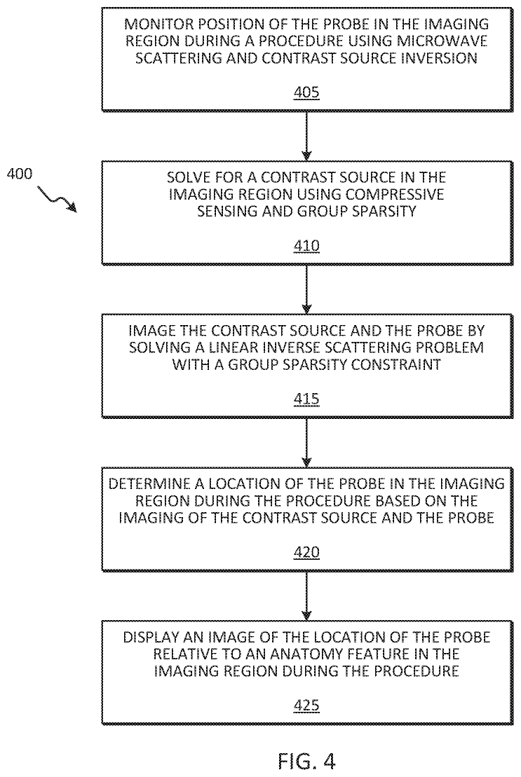

FIG. 4 is a flow chart of an example method 400 for tracking a probe during a procedure. The method 400 is described in terms of the tracking system 100 illustrated in FIG. 1. At block 405, the position of the probe 130 in the imaging region 120 is monitored using microwave scattering and contrast source inversion. At block 410, the tracking system 100 solves (for example, with the electronic processor 135) for a contrast source in the imaging region 120 using compressive sensing and group sparsity. The contrast source exists either at a surface of the probe 130 or within the probe 130. At block 415, the tracking system 100 images the contrast source and the probe 130 (for example, with the electronic processor 135) by solving a linear inverse scattering problem with a group sparsity constraint. In some embodiments, the electronic processor 135 solves the linear inverse scattering problem using a spectral gradient projection and a separable approximation. At block 420, the tracking system 100 determines (for example, with the electronic processor 135) a location of the probe 130 in the imaging region 120 during the procedure based on the imaging of the contrast source and the probe 130. In some embodiments, the electronic processor 135 also determines a three-dimensional shape of the probe 130 during the procedure based on the imaging of the contrast source and the probe 130. At block 425, the tracking system 100 displays (for example, on the display screen 150) an image of the location of the probe 130 relative to an anatomy feature in the imaging region 120 during the procedure. For example, the display screen 150 may display an image of the location of the probe 130 relative to a brain lesion.

In some embodiments, the position of the probe 130 is monitored in part by determining a plurality of scattering parameters by measuring a scattered electric field in the imaging region 120 with the vector network analyzer 110. In some embodiments, the computer 115 (or the vector network analyzer 110) links a plurality of total electric fields in the imaging region 120 excited by one or more transmitting antennas to the plurality of scattering parameters measured at one or more receiving antennas with an arbitrary inhomogeneous imaging background. In some embodiments, the position of the probe 130 is monitored in part by transmitting an excitation signal with a transmitting antenna into the imaging region 120 to produce an electric field in the imaging region 120.

Numerical Simulation

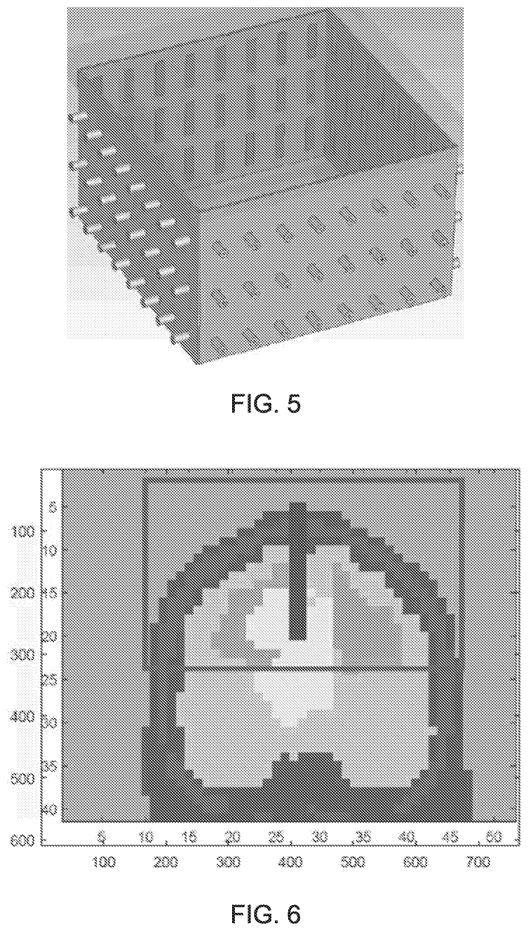

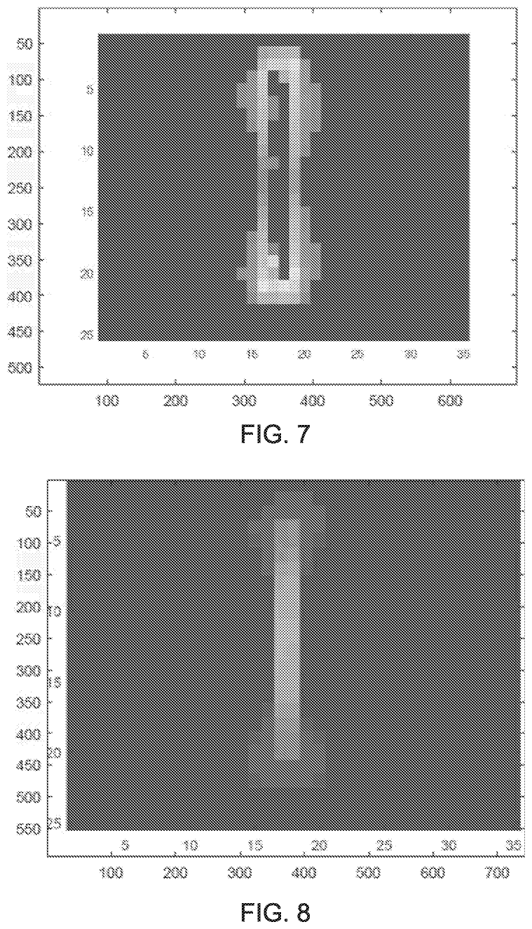

The microwave probe tracking method implemented by the tracking system 100 can be validated numerically by imaging the probe 130 while it is inserted in a realistic human head phantom. The head 125 located within the imaging region 120 in FIG. 1 is an exemplary human head phantom, derived from the Visible Human Project of NIH [7]. The imaging region 120 illustrated in FIG. 1 has a size of 15×25×15 cm³ and is used to contain the head 125 and collect scattering data of the probe 130. In some embodiments, as illustrated in FIG. 5, the imaging region 120 includes of ninety-six rectangular patching antennas working at 880 MHz, with forty-eight antennas being used as transmitters (i.e., transmitting antennas) and the other forty-eight antennas being used as receivers (i.e., receiving antennas). The finite difference time domain (FDTD) method can be used to simulate the scattered S-parameters produced by the contrast source of the probe 130. In some embodiments, the probe 130 is a pulsed eddy current (PEC) probe. The electronic processor 135 of the computer 115 reconstructs the three-dimensional shape and location of the probe 130 by solving the linear contrast source inversion problem. FIGS. 6, 7, and 8 illustrate examples of simulation imaging results. FIG. 6 illustrates an example simulation imaging result of a probe inserted into an exemplary human head model. FIG. 7 illustrates an example simulation imaging result of a metallic probe (for example, a Teflon PEC probe) inserted into a brain phantom. FIG. 8 illustrates an example simulation imaging result of a dielectric probe inserted into a brain phantom.

Experimental Validation

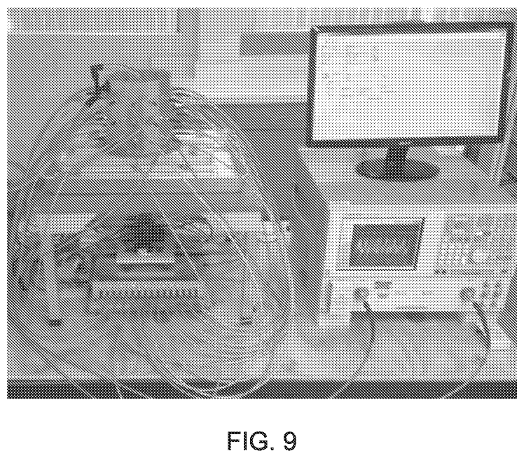

The probe tracking method described herein can be validated experimentally with a vector network analyzer-based inverse scattering measurement system. FIG. 9 illustrates an example measurement system including an imaging region with thirty-six patch antennas working at 900 MHz; with eighteen antennas being used as transmitters and the other eighteen antennas being used as receivers. The patch antennas are connected to a two-port vector network analyzer through a switching matrix, which creates three-hundred and twenty-four measurement paths with different transmitter/receiver antenna pairs. The imaging region is filled with an emulsion to mimic the dielectric properties of a human brain. MATLAB® is used to control the measurement system and process the inversion. Imaging results are generated with the probe placed at different locations within the imaging region.

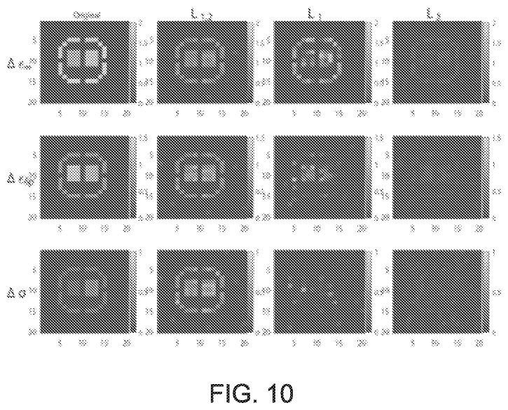

In some embodiments, the same framework described herein is used for multi-parameter contrast source microwave inverse scattering. In such embodiments, the group sparsity structure of individual parameters can be used to recover multiple unknowns with improved accuracy. As shown in FIG. 10, the second column of FIG. 10 shows the images of the three parameters reconstructed with our proposed approach. Compared with other methods, the group sparsity regularized approach described herein can effectively exploit the prior knowledge that each parameter's image has similar structure and can produce a more accurate shape and contrast recovery for each individual parameter with less computation time.

The numerical experiment setup for the results illustrated in FIG. 10 is as described below. A numerical study of imaging dispersive objects described by single-pole Debye models in [2] with three unknown parameters is presented. The original images are shown in the first column of FIG. 10, and the three parameters' images have the same structure. Twenty-seven transmitting antennas and twenty-seven receiving antennas are placed around the imaging region to take scattered field measurements at 1 GHz and 1.5 GHz. The imaging domain has 20×20×20 cubic pixels with a pixel edge length of 1.5 centimeters. As there are three unknown parameters per pixel, the total number of unknowns is 3×20×20×20=24,000. The total number of measurements is 27×27×2=1,458. The finite difference time domain method described herein can be used to solve the forward problem.

A comparison of the three regularization methods is illustrated below in Table 1. In Table 1, errₛ is the cost function error of the measured scattered field and can be determined as errₛ=∥Ŷ−Ā\hat{X}∥₂². Pₑᵣᵣ is the average pixel error within the image.

TABLE 1
Comparison of Three Regularization Methods

| errₛ = 1% | Pₑᵣᵣ | Time |
|---|---|---|
| l_{1, 2} | 3.1 × 10⁻⁴ | 36 seconds |
| l₁ | 4.0 × 10⁻⁴ | 90 seconds |
| l₂ | 5.4 × 10⁻⁴ | 81 seconds |

Thus, embodiments described herein relate to, among other things, real-time tracking of a probe during a procedure. For example, contrast source inversion-based microwave imaging systems and methods to track a probe during a medical procedure are disclosed herein. As the contrast source may only exist on the surface of the probe, compressive sensing is used in some embodiments to add a sparsity constraint to the problem. In some embodiments, a sparse solution of the formulated lasso problem is obtained with a spectral projected gradient method. It should be understood that the methods and systems described herein may be used to track various types of probes, including treatment probes, dielectric treatment probes, and the like.

It should be understood that the methods and systems described herein may be used to track a probe in a non-medical procedure. For example, the methods and systems described herein may be used to track a probe in a fabrication procedure or a compliance testing procedure.

The following references are incorporated herein by reference in their entirety.
[1] R. Kleinman and P. den Berg, “Two-dimensional location and shape reconstruction,” Radio Science, vol. 29, no. 4, pp. 1157-1169, 1994.
[2] G. Chen, J. Stang, and M. Moghaddam, “Numerical vector green's function for s-parameter extraction with waveport excitation,” submitted to IEEE Transactions on Antennas and Propagation, December 2016.
[3] H. Luyen, F. Gao, S. C. Hagness, and N. Behdad, “Microwave ablation at 10.0 GHz achieves comparable ablation zones to 1.9 GHz in ex vivo bovine liver,” IEEE Transactions on Biomedical Engineering, vol. 61, no. 6, pp. 1702-1710, June, 2014.
[4] R. Tibshirani, “Regression shrinkage and selection via the lasso”, Journal of Royal Statistical Society: Series B, vol 58, 1996.
[5] E. Berg and M. Friedlander, “Probing the pareto frontier for basis pursuit solutions”, SIAM Journal on Scientific computing, vol 31, 2008.
[6] S. Wright, R. Nowak and M. Figueiredo, “Sparse reconstruction by separable approximation”, IEEE Transactions on Signal Processing, vol 57, no. 7, July 2009
[7] NIH, “The national library of medicines visible human project.” [Online]. Available: https://www.nlm.nih.gov/research/visible/visible_human.html.

The components, steps, features, objects, benefits, and advantages that have been discussed are merely illustrative. None of them, nor the discussions relating to them, are intended to limit the scope of protection in any way. Numerous other embodiments are also contemplated. These include embodiments that have fewer, additional, and/or different components, steps, features, objects, benefits, and/or advantages. These also include embodiments in which the components and/or steps are arranged and/or ordered differently.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

All articles, patents, patent applications, and other publications that have been cited in this disclosure are incorporated herein by reference.

In this disclosure, the indefinite article “a” and phrases “one or more” and “at least one” are synonymous and mean “at least one.”

The scope of protection is limited solely by the claims that now follow. That scope is intended and should be interpreted to be as broad as is consistent with the ordinary meaning of the language that is used in the claims when interpreted in light of this specification and the prosecution history that follows, except where specific meanings have been set forth, and to encompass all structural and functional equivalents.

Relational terms such as “first” and “second” and the like may be used solely to distinguish one entity or action from another, without necessarily requiring or implying any actual relationship or order between them. The terms “comprises,” “comprising,” and any other variation thereof when used in connection with a list of elements in the specification or claims are intended to indicate that the list is not exclusive and that other elements may be included. Similarly, an element proceeded by an “a” or an “an” does not, without further constraints, preclude the existence of additional elements of the identical type.

None of the claims are intended to embrace subject matter that fails to satisfy the requirement of Sections 101, 102, or 103 of the Patent Act, nor should they be interpreted in such a way. Any unintended coverage of such subject matter is hereby disclaimed. Except as just stated in this paragraph, nothing that has been stated or illustrated is intended or should be interpreted to cause a dedication of any component, step, feature, object, benefit, advantage, or equivalent to the public, regardless of whether it is or is not recited in the claims.

The abstract is provided to help the reader quickly ascertain the nature of the technical disclosure. It is submitted with the understanding that it will not be used to interpret or limit the scope or meaning of the claims. In addition, various features in the foregoing detailed description are grouped together in various embodiments to streamline the disclosure. This method of disclosure should not be interpreted as requiring claimed embodiments to require more features than are expressly recited in each claim. Rather, as the following claims reflect, inventive subject matter lies in less than all features of a single disclosed embodiment. Thus, the following claims are hereby incorporated into the detailed description, with each claim standing on its own as separately claimed subject matter.

Various embodiments and features are set forth in the following claims.

What is claimed is:

1. A method for tracking a probe during a procedure, the method comprising:
monitoring a position of the probe in an imaging region during the procedure using microwave inverse scattering and contrast source inversion;
solving for a contrast source in the imaging region using compressive sensing and group sparsity, wherein the contrast source exists at a surface of the probe or within the probe;
imaging the contrast source and the probe by solving a linear inverse scattering problem with a group sparsity constraint;
determining a location of the probe in the imaging region during the procedure based on the imaging of the contrast source and the probe; and
displaying an image of the location of the probe relative to an anatomy feature in the imaging region during the procedure.

2. The method of claim 1, further comprising determining a plurality of scattering parameters by measuring a scattered electric field in the imaging region with a vector network analyzer.

3. The method of claim 2, further comprising linking a plurality of total electric fields in the imaging region excited by one or more transmitting antennas to the plurality of scattering parameters measured at one or more receiving antennas with an arbitrary inhomogeneous imaging background.

4. The method of claim 1, wherein solving the linear inverse scattering problem includes solving the linear inverse scattering problem using a spectral gradient projection and a separable approximation.

5. The method of claim 1, furthering comprising determining a three-dimensional shape of the probe during the procedure based on the imaging of the contrast source and the probe.

6. The method of claim 1, wherein the contrast source is defined in part as a product of a material contrast and a total electric field in the imaging region.

7. The method of claim 1, further comprising transmitting an excitation signal with a transmitting antenna into the imaging region to produce an electric field in the imaging region.

8. The method of claim 1, wherein the probe is a metallic probe, and wherein, within the imaging region, the contrast source only exists at the surface of the probe.

9. The method of claim 1, wherein the probe is a non-metallic probe, and wherein, within the imaging region, the contrast source only exists within the probe.

10. The method of claim 1, wherein solving the linear inverse scattering problem includes solving the linear inverse scattering problem with a graphics processing unit.

11. A system for tracking a probe during a procedure, the system comprising:
a plurality of antennas arranged in a three-dimensional array in an imaging region, the plurality of antennas configured to generate a contrast source by exciting a medium of the probe during the procedure; and
a computer operatively connected to the plurality of antennas, the computer including:
an electronic processor configured to
determine scattering data generated by the contrast source based on a plurality of measurements from the plurality of antennas,
determine a linear inverse scattering solution including a group sparsity constraint based on the scattering data,
image the contrast source and the probe based on the linear inverse scattering solution, and
determine a location of the probe in the imaging region during the procedure based on the imaging of the contrast source and the probe; and
a display screen configured to display the location of the probe relative to an anatomy feature in the imaging region during the procedure.

12. The system of claim 11, further comprising a vector network analyzer operatively connected to the plurality of antennas and the computer, wherein the vector network analyzer is configured determine a plurality of scattering parameters based on the plurality of measurements from the plurality of antennas, wherein the plurality of scattering parameters indicating a scattered electric field measured in the imaging region.

13. The system of claim 12, wherein the plurality of antennas including one or more transmitting antennas and one or more receiving antennas, wherein the vector network analyzer is further configured to link a plurality of total electric fields in the imaging region generated by the one or more transmitting antennas to the plurality of scattering parameters measured at the one or more receiving antennas with an arbitrary inhomogeneous imaging background.

14. The system of claim 11, wherein the electronic processor is configured to determine the linear inverse scattering solution using a spectral gradient projection and a separable approximation.

15. The system of claim 11, wherein the electronic processor is further configured to determine a three-dimensional shape of the probe during the procedure based on the imaging of the contrast source and the probe.

16. The system of claim 11, wherein the contrast source is defined in part as a product of a material contrast and a total electric field in the imaging region.

17. The system of claim 11, wherein the plurality of antennas including one or more transmitting antennas configured to transmit an excitation signal into the imaging region to produce an electric field in the imaging region.

18. The system of claim 11, wherein the medium of the probe includes a metal disposed on a surface of the probe, and wherein the contrast source only exists at the surface of the probe.

19. The system of claim 11, wherein the medium of the probe includes a non-metal disposed within the probe, and wherein the contrast source only exists within the probe.

20. The system of claim 11, wherein the electronic processor includes a graphics processing unit, wherein the electronic processor is configured to determine the linear inverse scattering solution with the graphics processing unit.
